(19) United States
(12) Patent Application Publication
(10) Pub. No.: US 2019/0256819 A1
(43) Pub. Date: Aug. 22, 2019

(54) PROCESS FOR IMPROVING THE DISSOLUTION BEHAVIOUR OF COMPONENTS IN AQUEOUS SOLUTIONS

(71) Applicant: Merck Patent GmbH, Darmstadt (DE)

(72) Inventors: Joerg VON HAGEN, Pfungstadt (DE); Andrew SALAZAR, Darmstadt (DE); Markus LUBDA, Darmstadt (DE)

(73) Assignee: Merck Patent GmbH, Darmstadt (DE)

(21) Appl. No.: 16/320,543

(22) PCT Filed: Jul. 25, 2017
(86) PCT No.: PCT/EP2017/068727

§ 371 Date: Jan. 25, 2019

(30) Foreign Application Priority Data
Jul. 28, 2016 (EP) 16181691.3

(51) Int. Cl. C12N 5/00

(52) U.S. Cl. CPC C12N 5/0018; C12N 2500/32; C12N 2500/14; C12N 2500/34; C12N 2500/12; C12N 2500/38; C12N 2500/46; C12N 2500/16; C12N 2500/36

(57) ABSTRACT

The present invention relates to a process for improving the solubility or dissolution behaviour of components in an aqueous solution. The dissolution properties of a slowly/badly dissolving component can be positively affected by generating mixed particles of this component with another component which has a negative dissolution enthalpy. By preparing and adding such mixed particles solubility is improved without changing the chemical composition.

The present invention relates to a process for improving the solubility or dissolution behaviour of components in an aqueous solution. The dissolution properties of a slowly/badly dissolving component can be positively affected by generating mixed particles of this component with another component which has a negative dissolution enthalpy. By preparing and adding such mixed particles solubility is improved without changing the chemical composition.

### BACKGROUND OF THE INVENTION

When preparing dry powder mixtures or other dry mixtures of components that shall be easily and fully soluble in aqueous solutions, one often faces the problem that certain components are not fully dissolved or can only be dissolved after a long time of mixing compared to the mixing time required for the rest of the components. Examples for such dry mixtures are dry powder cell culture media, nutrient compositions e.g. effervescent tablets, dry mixtures for the preparation of energy drinks, solutions for infusion etc.

Those dry mixtures might be in the form of a dry powder, a compacted, solid form or any type of tablet. The mixtures typically comprise several different ingredients like saccharide components, amino acids, vitamins or vitamin precursors, salts, buffer components, co-factors and/or nucleic acid components.

One example of such a complex dry mixture are cell culture media. Cell culture media in aqueous solution can provide an environment which supports and maintains the growth of cells and/or maintains a desired physiological cellular condition advantageous to the targeted production of certain products.

Cell culture media comprise of a complex mixture of components, sometimes more than one hundred different components, depending on the type of organism whose growth and/or targeted physiological status shall be supported.

The cell culture media required for the propagation of mammalian, insect or plant cells are typically much more complex than the media to support the growth of bacteria, yeast or fungi.

The first cell culture media that were developed consisted of undefined components, such as plasma, serum, embryo extracts, or other non-defined biological extracts or peptones. A major advance was thus made with the development of chemically defined media. Chemically defined media often comprise but are not exclusively limited to amino acids, vitamins, metal salts, antioxidants, chelators, growth factors, buffers, hormones, and many more substances known to those expert in the art.

Some cell culture media are offered as sterile aqueous liquids. The disadvantage of liquid cell culture media is their reduced shelf life and difficulties for shipping and storage. As a consequence, many cell culture media are presently offered as finely milled dry powder mixtures. They are manufactured for the purpose of dissolving in water and/or aqueous solutions and in the dissolved state are designed, often with other supplements, for supplying cells with a substantial nutrient base for growth and/or production of biopharmaceuticals from same said cells.

Most biopharmaceutical production platforms are based on fed-batch cell culture protocols. The aim typically is to develop high-titer cell culture processes to meet increasing market demands and reduce manufacturing costs. Beside the use of high-performing recombinant cell lines, improvements in cell culture media and process parameters are required to realize the maximum production potentials.

In a fed-batch process, a basal medium supports initial growth and production, and a feed medium prevents depletion of nutrients and sustains the production phase. The media are chosen to accommodate the distinct metabolic requirements during different production phases. Process parameter settings—including feeding strategy and control parameters—define the chemical and physical environments suitable for cell growth and protein production.

Optimization of the feed medium is major aspect in the optimization of a fed-batch process.

Mostly the feed medium is highly concentrated to avoid dilution of the bioreactor. The controlled addition of the nutrient directly affects the growth rate of the culture.

U.S. Pat. No. 6,383,810 B2 by Invitrogen Corporation discloses a method of producing an agglomerated eukaryotic cell culture medium powder. The method comprises wetting a dry powder cell culture medium with a solvent and then re-drying the moistened medium to obtain a dry agglomerated cell culture medium with potentially improved solubility.

A great disadvantage of this procedure is the fact that all medium components need to be contacted with water and that they need to be heated to remove the water afterwards. This may cause a change in the composition as the water content might change. In addition, it might cause considerable side reactions among the components of the medium or destruction or modification of sensitive components with an unpredictable outcome on the medium quality.

Nevertheless a limiting factor for the preparation of cell culture media from dry powder is the poor solubility of some components like amino acids. See also Salazar A, Keusgen M, Hagen J Von. Amino acids in the cultivation of mammalian cells. Amino Acids. 2016;48(5)1161-1171. doi:10.1007/s00726-016-2181-8. Consequently it would be favourable to find a way to improve the solubility of such compounds in cell culture media without negatively affecting the other components and without changing the composition of the medium. The same holds true for other dry mixtures like nutrient mixtures for humans or animals that are prepared by dissolving a dry component mixture in an aqueous solution.

### BRIEF DESCRIPTION OF THE INVENTION

It has been found that the solubility of compounds can be improved by generating mixed particles comprising the component whose solubility shall be improved in close proximity with at least one component whose dissolution enthalpy is negative. Those mixed particles are prepared by processes like spray drying or co-lyophilisation and can then be mixed with the other components to generate the final dry mixture. It has been found that when dissolving said mixture in an aqueous solvent, the solubility of the poorly dissolving component is improved. Assumably this is due to the fact that the energy which is set free by the component having a negative dissolution enthalpy can be at least partly transferred to the poorly soluble component which is—due to the mixed particles—in close proximity to the component with the negative dissolution enthalpy. This solubility improvement is achieved without changing the chemical composition of the cell culture medium and without applying other external means like heating, sonification or more vigorous stirring. It has been found that the inventive process is especially suitable for improving the solubility of amino acids by preparing mixed particles of at least one amino acids with a negative enthalpy of solution and at least one amino acid with a positive enthalpy of solution.

The present invention is thus directed to a method for improving the solubility of a dry mixture with a given composition by
a) identifying the one or more poorly soluble components in the said dry mixture
b) preparing mixed particles comprising at least one poorly soluble component and one component with a negative enthalpy of solution, whereby also the component with a negative enthalpy of solution is selected from the components present in the given composition of the dry mixture
c) preparing the said dry mixture by mixing the one or more mixed particles generated in step b) with the other components of the dry mixture

Preferably, the other components with whom the mixed particles are mixed in step c) are present in the form of particles of single components and are not present in the form of component mixtures prepared by spray drying, wet granulation, co-lyophilisation or other techniques involving dissolving and re-drying all or a large part of the components. An exception would be the presence of a second type of mixed particles according to the invention in case the solubility of more than one poorly soluble components shall be improved. Preferably, more than 70% (w/w) of the mixture components are present in the form of single component particles.

In a preferred embodiment, the one or more poorly soluble components are compounds with a solubility of less than 20 g in an aqueous solution comprising more than 100 g/l of dissolved components at 25° C. Preferably, poorly soluble components are components with a positive enthalpy of solution.

In a preferred embodiment, the one or more components with a negative enthalpy of solution are selected from the following group:
proline
Calcium Chloride, anhydrous
Magnesium Sulfate, anhydrous
Calcium Oxide, anhydrous
Sodium Hydroxide
Potassium Hydroxide

or mixtures thereof

Most preferred component with a negative enthalpy of solution is proline.

In a preferred embodiment, the one or more poorly soluble components are selected from the group of leucine, isoleucine, valine and phenylalanine.

In another preferred embodiment in step b) the preparation of the mixed particles is performed by spray drying, lyophilisation or wet granulation.

In a most preferred embodiment in step b) the preparation of the mixed particles is performed by spray drying.

Very preferred the mixed particles generated in step b) do not contain more than 10% (w/w) of components which are either not poorly soluble or do not have a negative enthalpy of solution. That means that preferably 90% or more (w/w) of the mixed particles consists of the one or more poorly soluble components and the one or more components with a negative enthalpy of solution.

Very preferred the mixed particles generated in step b) only contain amino acids.

In a preferred embodiment, the dry mixture is a dry powder cell culture medium.

In a preferred embodiment, the composition of the mixed particles is such that the overall amount of the negative enthalpy of solution resulting from dissolution of the mixed particle is double or more than the amount of the positive enthalpy of solution resulting from dissolving the poorly soluble component present in the mixed particle.

In a preferred embodiment, the mixed particles prepared in step b) comprise proline in combination with one or more of the components selected from leucine, isoleucine, valine and phenylalanine.

The present invention is further directed to dry mixtures comprising one or more mixed particles containing at least one component which is poorly soluble in water and at least one component with a negative dissolution enthalpy. Preferably, the mixed particles only contain one component which is poorly soluble in water and one component with a negative dissolution enthalpy.

In a preferred embodiment, the dry mixture is a dry powder cell culture medium.

In another preferred embodiment the dry mixture comprises mixed particles comprising proline in combination with one or more of the components selected from leucine, isoleucine, valine and phenylalanine.

### DESCRIPTION OF THE INVENTION

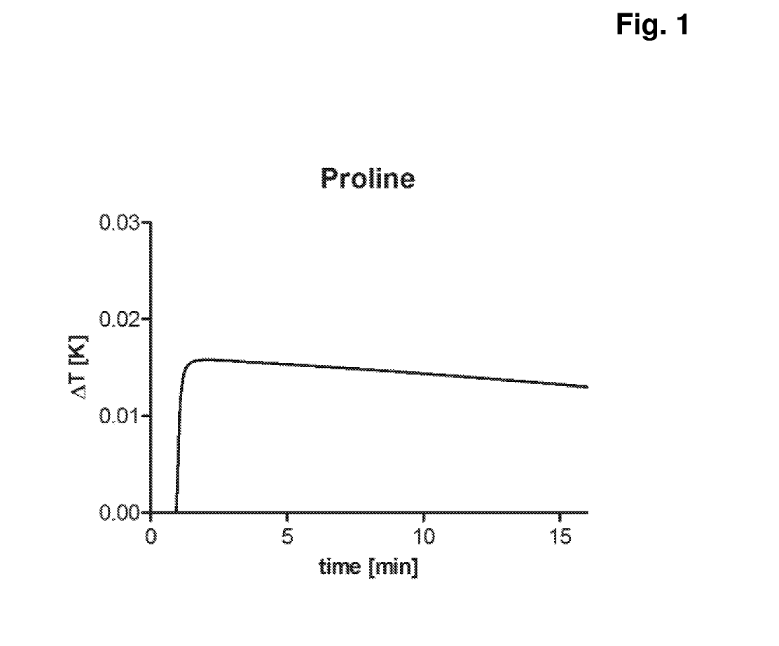

FIG. 1 shows the relative temperature as a function of time after correction of the initial baseline slope of L-proline. Values represent mean (n=2).

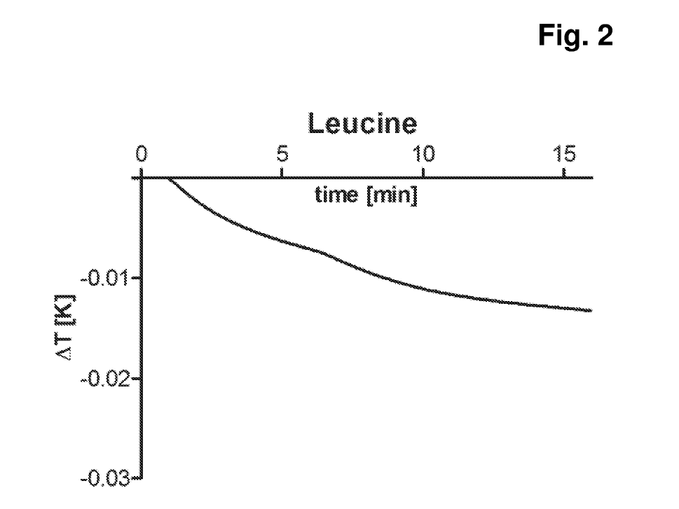

FIG. 2: Relative temperature as a function of time after correction of the initial baseline slope of L-leucine. Values represent mean (n=2).

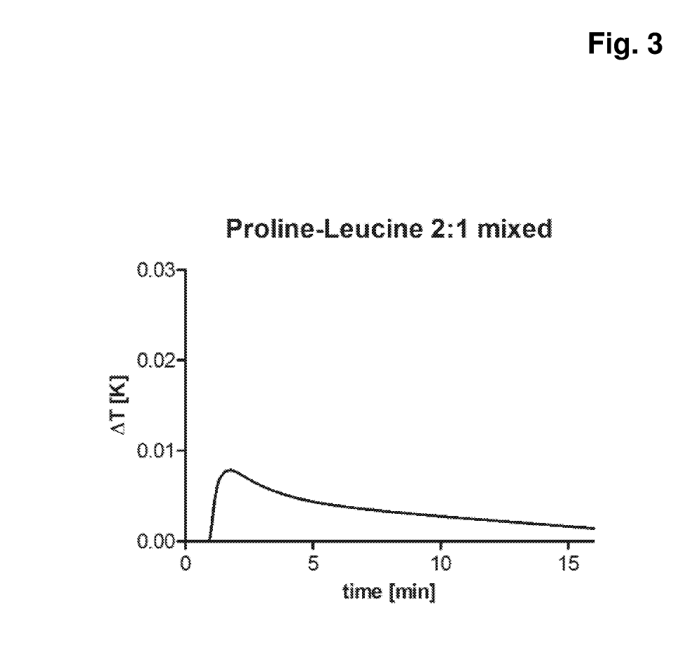

FIG. 3 Relative temperature as a function of time after correction of the initial baseline slope of mixed L-proline and L-leucine powder in the ratio 2:1. Values represent mean (n=3).

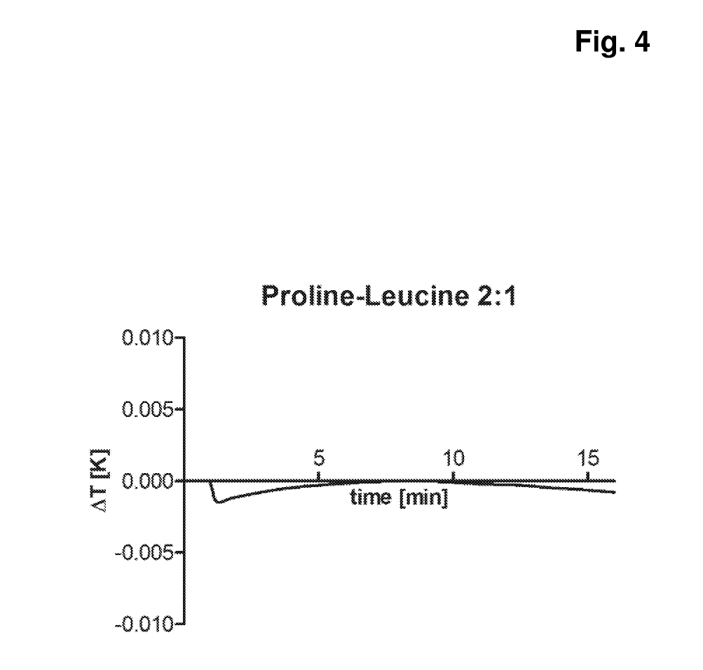

FIG. 4: Relative temperature as a function of time after correction of the initial baseline slope of spry dried L-proline and L-leucine powder in the ratio 2:1. Values represent mean (n=3).

Further details about the Figures can be found in Example 3.

A dry mixture according to the present invention is any mixture of components that comprises two or more components selected from one or more groups of the following: saccharide components, amino acids, vitamins or vitamin precursors, salts, buffer components, co-factors and/or nucleic acid components. Dry mixtures according to the present invention are dry mixtures meant for complete dissolution in an aqueous solution. An aqueous solution is water or any aqueous solution like buffers. The aqueous solution might comprise up to 20% of a water soluble organic solvent like ethanol. But preferably the solvent is water or an aqueous buffer and does not contain any organic solvent.

Typically the dry mixture of components is in the form of a dry powder, any compacted form of a dry powder or in the form of tablets.

The term “dry powder” may be used interchangeably with the term “powder;” however, “dry powder” as used herein simply refers to the gross appearance of the granulated material and is not intended to mean that the material is completely free of complexed or agglomerated solvent unless otherwise indicated.

Preferably all components of the dry mixture are dry. That means, if they comprise water, they do only comprise water of crystallization but not more than 5%, preferably not more than 2% most preferred not more than 1% by weight of unbound or uncoordinated water molecules.

A powdered cell culture medium can be a cell culture medium typically resulting from a milling process or a lyophilisation process. It can also be a granulated cell culture medium, e.g. dry granulated by roller compaction.

The dry mixture according to the present invention can be any mixture of components meant for the preparation of liquid cell culture media or other liquid compositions like nutrient/vitamin drinks, isotonic drinks, solutions for infusion etc.. examples for nutrient mixtures can be found in US2008/0254119. Preferably, the dry mixtures are dry powder cell culture media.

Mixed particles are particles comprising at least one poorly soluble component and one component with a negative enthalpy of solution whereby all components of the mixed particle are mixed and distributed within the particle as thoroughly and as homogenously as possible to enlarge the interacting interface between the poorly soluble components and the components with a negative enthalpy of solution. The size of the mixed particles is comparable to the particle size of the other components of the dry mixture. Typical particle sizes are below 200 μm, preferably below 100 μm. Preferred are particle sizes between 15 μm and 100 μm. The better the mixing and blending of the components in the mixed particle the better. Mixed particles can be prepared by any means of preparing particles of a mixture of components. This can be by dry compaction of powder mixtures, or preferably by wet granulation, spray drying or lyophilisation, especially preferred is spray-drying.

Dry compaction is typically performed in two basic steps.
a) providing a mixture of components in form of a mixed powder of the components
b) compacting said mixed powder in a roll press

In step a), a finely milled dry powder of the components is provided. All components are thoroughly mixed so that all parts of the powder mixture have nearly the same composition. The mixture should have the same quality with respect to its uniformity of composition as a commercial dry powder cell culture medium. The higher the uniformity of the composition, the better the quality of the resulting dry granulated mixture. Mills which are suitable to produce fine milled powders are for example ball mills, pin mills, jet mills or rotating blade sieve mills. The particle size of the powders is typically below 500 μm, preferably below 200 μm.

A roll press, also called roller compactor, is known to a person skilled in the art. Typically a roll press comprises two counter-rotating rolls which are located at a small distance of about 0.5 to 3 mm, preferably 1 to 2 mm next to each other. Suitable roll presses typically have rolls with a widths between 10 and 50 cm resulting in the gap between the roll having a length between 10 and 50 cm. Nevertheless, the size of the rolls and thus the length of the gap between the rolls can vary depending on the size of the roll press. In a preferred embodiment, the gap has a length between 10 and 15 cm.

The mixed powder material is drawn in between the counter-rotating rolls and compacted in the gap between the rolls. The distance between the rolls and their surface structure have influence on the final size and structure of the resulting granulated particles.

Lyophilisation according to the present invention is freeze-drying by freezing the material and then reducing the surrounding pressure to allow the frozen water and optionally other volatile components in the material to sublimate directly from the solid phase to the gas phase.

As used herein, “co-lyophilised” or “co-lyophilisate” refers to a product resulting from the lyophilization, freeze-drying, cryodesiccation or vacuum drying of more than one compound in solution in the same vessel. For example, two solutions might be combined in the same vessel and the resulting combination of solutions is lyophilized together, thereby lyophilizing the components in the solutions simultaneously. Alternatively, two or more compounds can be dissolved in the same liquid and afterwards be lyophilised together. The resulting product of such a co-lyophilisation is a co-lyophilisate consisting of solid material that comprises a mixture of all non-volatile components that have been co-lyophilised.

If the mixed particles are generated by spray drying, a concentrated solution of all components to be mixed is generated, containing the components in the ratio desired. The solution is pumped into the chamber of a spray dryer, where it is dispersed and the solvent is evaporated using hot air. The conjugated material is collected from the collection chamber and can be used without further processing.

In wet granulation, granules or mixed particles of the components are formed by the addition of a granulation liquid onto a powder bed which is under the influence of an impeller (in a high-shear granulator), screws (in a twin screw granulator) or air (in a fluidized bed granulator). The agitation resulting in the system along with the wetting of the components within the formulation results in the aggregation of the primary powder particles to produce wet granules. The granulation liquid contains a solvent which must be volatile so that it can be removed by drying, and be non-toxic. Typical liquids include water, ethanol and isopropanol either alone or in combination.

A cell culture medium according to the present invention is any mixture of components which maintains and/or supports the in vitro growth of cells and/or supports a particular physiological state. It might be a complex medium or a chemically defined medium. The cell culture medium can comprise all components necessary to maintain and/or support the in vitro growth of cells or only some components so that further components are added separately. Examples of cell culture media according to the present invention are full media which comprise all components necessary to maintain and/or support the in vitro growth of cells, media supplements or feeds. In a preferred embodiment the cell culture medium is a full medium or a feed.

Typically, the cell culture media according to the invention are used to maintain and/or support the growth of cells in a bioreactor and/or to support a particular physiological state.

A mammalian cell culture medium is a mixture of components which maintain and/or support the in vitro growth of mammalian cells. Examples of mammalian cells are human or animal cells, preferably CHO cells, COS cells, I VERO cells, BHK cells, AK-1 cells, SP2/0 cells, L5.1 cells, hybridoma cells or human cells. Suitable cell culture methods are for example fed batch processes or perfusion cell culture processes.

Chemically defined cell culture media are cell culture media that do not comprise any chemically undefined substances. This means that the chemical composition of all the chemicals used in the media is known. The chemically defined media do not comprise any yeast, animal or plant tissues; they do not comprise feeder cells, serum, extracts or digests or other components which may contribute chemically poorly defined proteins to the media. Chemically undefined or poorly defined chemical components are those whose chemical composition and structure is not known, are present in varying composition or could only be defined with enormous experimental effort—comparable to the evaluation of the chemical composition and structure of a protein like albumin or casein.

A powdered cell culture medium is a cell culture medium resulting from a milling process. That means the powdered cell culture medium is a dry, particulate medium—not a liquid medium.

Cells to be cultured with the media according to the present invention may be prokaryotic cells like bacterial cells or eukaryotic cells like plant or animal cells. The cells can be normal cells, immortalized cells, diseased cells, transformed cells, mutant cells, somatic cells, germ cells, stem cells, precursor cells or embryonic cells, any of which may be established or transformed cell lines or obtained from natural sources.

An inert atmosphere is typically generated by filling the respective container or apparatus with an inert gas. Suitable inert gases are noble gases like argon or preferably nitrogen. These inert gases are non-reactive and prevent undesirable chemical reactions from taking place. According to the present invention, generating an inert atmosphere means that the concentration of oxygen is reduced below 10% (v/v) absolute, e.g. by introducing liquid nitrogen or nitrogen gas.

Different types of mills are known to a person skilled in the art.

A pin mill, also called centrifugal impact mill, pulverizes solids whereby protruding pins on high-speed rotating disks provide the breaking energy. Pin mills are for example sold by Munson Machinery (USA), Premium Pulman (India), Hosokawa Alpine or Sturtevant (USA).

A jet mill uses compressed gas to accelerate the particles, causing them to impact against each other in the process chamber. Jet mills are e.g. sold by Sturtevant (USA), Hosokawa Alpine or PMT (Austria).

A fitz mill commercialized by Fitzpatrick (USA), uses a rotor with blades for milling.

The cell culture media typically comprise at least one or more saccharide components, one or more amino acids, one or more vitamins or vitamin precursors, one or more salts, one or more buffer components, one or more co-factors and one or more nucleic acid components.

The media may also comprise sodium pyruvate, insulin, vegetable proteins, fatty acids and/or fatty acid derivatives and/or pluronic acid and/or surface active components like chemically prepared non-ionic surfactants. One example of a suitable non-ionic surfactant are difunctional block copolymer surfactants terminating in primary hydroxyl groups also called poloxamers, e.g. available under the trade name pluronic ® from BASF, Germany. Such substances are block copolymers based on ethylene oxide and propylene oxide (Polyethylene glycol (PEG)/polypropylene glycol (PPG) block copolymers). The poloxamers, CAS number 9003-11-6, to be preferably used have the general formula I

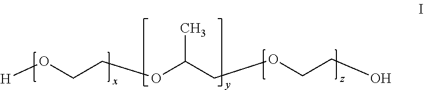

with x and z preferably independently being 2 to 130 and y preferably being 15 to 67.

Commercially available poloxamers are e.g. Pluronics® or Lutrole®, e.g., a Pluronic(R) solution, gel, or solid, such as Pluronic(R) F-127). Alternatively, the poloxamer can be made from raw materials according to methods known in the art (see, for example, U.S. Pat. Nos. 3,579,465 and 3,740,421).

Particularly preferred is Poloxamer 188 sometimes called Pluronic F 68 or Kolliphor P 188 or Lutrol F 68.

Saccharide components are all mono- or di-saccharides, like glucose, galactose, ribose or fructose (examples of monosaccharides) or sucrose, lactose or maltose (examples of disaccharides).

Examples of amino acids or amino acid components according to the invention are the proteinogenic amino acids, especially the essential amino acids, leucine, isoleucine, lysine, methionine, phenylalanine, threonine, tryptophane and valine, as well as the non-proteinogenic amino acids like D-amino acids. Further examples are proline and valine.

Examples of vitamins are Vitamin A (Retinol, retinal, various retinoids, and four carotenoids), Vitamin B₁ (Thiamine), Vitamin B₂ (Riboflavin), Vitamin B₃ (Niacin, niacinamide), Vitamin B₅ (Pantothenic acid), Vitamin B₆ (Pyridoxine, pyridoxamine, pyridoxal), Vitamin B₇ (Biotin), Vitamin B₉ (Folic acid, folinic acid), Vitamin B₁₂ (Cyanocobalamin, hydroxycobalamin, methylcobalamin), Vitamin C (Ascorbic acid), Vitamin D (Ergocalciferol, cholecalciferol), Vitamin E (Tocopherols, tocotrienols) and Vitamin K (phylloquinone, menaquinones). Vitamin precursors are also included.

Examples of salts are components comprising inorganic ions such as bicarbonate, calcium, chloride, magnesium, phosphate, potassium and sodium or trace elements such as Co, Cu, F, Fe, Mn, Mo, Ni, Se, Si, Ni, Bi, V and Zn. Examples are Copper(II) sulphate pentahydrate (CuSO₄.5H₂O), Sodium Chloride (NaCl), Calcium chloride (CaCl₂.2H₂O), Potassium chloride (KCl), Iron(11)sulphate, sodium phosphate monobasic anhydrous (NaH₂PO₄), Magnesium sulphate anhydrous (MgSO₄), sodium phosphate dibasic anhydrous (Na₂HPO₄), Magnesium chloride hexahydrate (MgCl₂.6H₂O), zinc sulphate heptahydrate.

Examples of buffers are CO₂/HCO₃ (carbonate), phosphate, HEPES, PIPES, ACES, BES, TES, MOPS and TRIS.

Examples of cofactors are thiamine derivatives, biotin, vitamin C, NAD/NADP, cobalamin, flavin mononucleotide and derivatives, glutathione, heme nucleotide phophates and derivatives.

Nucleic acid components, according to the present invention, are the nucleobases, like cytosine, guanine, adenine, thymine or uracil, the nucleosides like cytidine, uridine, adenosine, guanosine and thymidine, and the nucleotides like adenosine monophosphate or adenosine diphosphate or adenosine triphosphate.

For all compound mentioned the compound name also includes any salts, hydrates, di-, tri- or oligomers or enantiomers. For example glucose also means glucose monohydrate, or Hepes (4-(2-hydroxyethyl) piperazine-1-ethanesulfonic acid) also means Hepes sodium salt (4-(2-Hydroxyethyl)piperazine-1-ethanesulfonic acid sodium salt).

Freezing according to the present invention means cooling to a temperature below 0° C.

The gist of the present invention is to improve the solubility of poorly soluble components of a dry mixture of components and thus the overall solubility of a mixture with a given composition. Preferred mixtures are cell culture media. Powdered cell culture media with a given composition might comprise one or several components which do not dissolve in a given amount of water under given dissolution conditions or need a special treatment like elevated temperature or a long time to dissolve. It is typically not desirable to amend the overall composition of the cell culture medium to reduce the amount of the poorly soluble component or substitute it by another component.

It has been found that with the method of the present invention the solubility of a powdered cell culture medium and other dry mixtures can be improved without changing their chemical composition.

This is done by identifying in a given composition the one or more components which are poorly soluble and hinder easy dissolution of the dry powder mixture.

A person skilled in the art of cell culture media it typically aware of the solubilities of the typical ingredients of cell culture media and can identify the poorly soluble components in a given list of ingredients.

Identification can also be done by reviewing the list of ingredients of a given composition and identifying the one or more components with the poorest solubility based on solubility data. The solubility of substances is typically known and can be found in publicly available data bases or text books, e.g. Chemiker-Kalender. editors Claudia Synowietz and Klaus Schäfer; 3. Edition, 2. Reprint 1992; 656 pages; Berlin, Heidelberg, New York, Tokyo; Springer 1984; ISBN 978-3-540-12652-2.

Identification can also be done experimentally by dissolving a given dry mixture and in case of incomplete dissolution, isolating the non-dissolved particles and analysing them e.g. by NMR, mass spectrometry, elementary analysis, chromatographic methods or combinations thereof. Examples of suitable methods are ICP-OES, UPLC (e.g. for amino acids), IC. From this analysis one obtains information about the chemical composition of the non-dissolved particles and can thus identify the poorly soluble components.

A component that is poorly soluble according to the present invention is any component of a mixture whose solubility in said mixture composition shall be improved under given dissolution conditions. Poorly soluble components are often described as substances of which less than 10 g, especially less than 1 g, can be dissolved in 1 liter of water at 25° C. The present invention is suitable for such poorly soluble substances but can of course also be used for substances of which more than 10 g can be dissolved in 1 liter of water at 25° C. if further improvement of their solubility is needed, for example if they need to be added in quite large amounts or if the time needed for dissolving the substance is comparably long. In addition, the solubility of a substance in water can differ from its solubility in a mixture comprising several other dissolved components. This is especially true for highly concentrated solutions like feed media. In solutions which contain more than 100 g/l of dissolved components, the solubility of components which have a solubility of more than 10 g/l in water at 25° C. might not be sufficient and even such components are poorly soluble under those conditions. This is e.g. the case for amino acids like leucine, valine, isoleucine and phenylalanine with a solubility of typically more than 10 g/l in pure water. Nevertheless, in highly concentrated feed media, their solubility is often not sufficient. Consequently, a substance that is poorly soluble according to the present invention is preferably a substance of which less than 20 g, especially less than 10 g, can be dissolved in 1 liter of an aqueous solution comprising more than 100 g/l of dissolved components or, as an example of such a solution, in ten-fold concentrated DMEM F12 cell culture medium at 25° C. The composition of one-fold DMEM F12 cell culture medium (product of Sigma Aldrich, article number D2906) is shown in the Examples. Typically the dissolution should be completed after 2 hours, preferably after 45 minutes.

Examples of poorly soluble cell culture media components are known to a person skilled in the art or are identified as described above for a given cell culture media composition. They may differ depending on the type of cell culture medium. Typical examples are: cystine, ferric citrate (iron (III) citrate hydrate), Fe(III) chloride hexahydrate and/or tyrosine. While cystine is poorly soluble in water and cell culture media, Fe(III) chloride hexahydrate is an example of a component which is sufficiently soluble in water but typically shows very poor solubility in cell culture media.

Preferred poorly soluble components according to the present invention are components having a positive enthalpy of solution. They can be saccharide components, amino acids, vitamins or vitamin precursors, salts, buffer components, co-factors and/or nucleic acid components. Examples of salts are potassium chloride and ammonium nitrate. Preferably the poorly soluble components with a positive enthalpy of solution are amino acids. Examples are leucine, isoleucine, valine and phenylalanine.

The enthalpy of solution, enthalpy of dissolution, or heat of solution is the enthalpy change associated with the dissolution of a substance in a solvent at constant pressure resulting in infinite dilution.

The enthalpy of solution is most often expressed change in kJ/mol at constant temperature. The energy can be regarded as being made of three parts, the endothermic breaking of bonds within the solute and within the solvent, and the formation of attractions between the solute and the solvent.

The energy change on dissolution, the enthalpy of solution, of a compound depends on its structure and the solvent in which it is dissolving. Some of this energy can also be transferred to other molecules in the solution, called enthalpy of transfer, and some is lost to the surroundings. It has been found that this effect can be used to improve the solution behavior of poorly soluble components. It seems that the enthalpy of transfer can improve the solubility of a poorly soluble component locally, on a molecular level as also the energy is transferred on molecular level without the need of energy transfer to the whole mixture e.g. by heating the whole mixture.

In the case of dissolving substances, components can be classified as exothermic or endothermic based on their release or uptake of energy. The thermal energy and the enthalpy is calculated, by using the formula (1) and

(2). Since measurements are made at high dilutions, the heat capacity of water can be used for aqueous solutions. The calculation is clarified with an example as follows:

$$\begin{matrix}Δ Q = m * {c}_{p} * Δ T & ( 1 ) \\ Δ H = \frac{Δ Q}{n} & ( 2 )\end{matrix}$$

ΔH=Enthalpy of reaction [kJ/mol]
ΔQ=change of thermal energy [kJ]
m=mass of substance [g]
cₚ=heat capacity of water, 4.18 [J/kg K]
ΔT=change of temperature [K]
n=amount of substance

### Example: Glycine was Added to 800 g of Water

$$m = m ( Glycine ) + m ( water ) \\ \to m = 6.82 g + 800 g$$

$$Δ T = 0.353 K$$

$$n = 0.09 mol$$

$$Δ Q = 806.82 g * 4.18 \frac{J}{kgK} * 0.353 K$$

$$Δ Q = 1.19 J$$

$$Δ H = \frac{1.19 J}{0.09 mol}$$

$$Δ H = 13.108 \frac{kJ}{mol}$$

Further details about the enthalpy of solution of amino acids can be found in Lou Y, Lin R. Enthalpy of transfer of amino acids from water to aqueous glucose solutions at 298 . 15 K. 1998;316:145-148, and Acta T. Enthalpies of solution of L-phenylalanine in aqueous solution of amides at 298 . 15 K. 2016;(January 2013). doi:10.1016/j.tca.2012.11.004.

It has been found that the energy transfer of a components with negative enthalpy of solution to a component with positive enthalpy of solution is sufficiently effective to support dissolution of the poorly soluble component if the components are in close proximity during dissolution. This close proximity can be achieved by the generation of mixed particle of those components. The poorly soluble components are consequently mixed with one or more components having a negative enthalpy of solution to form mixed particles prior to adding them to the dry powder mixture. It is possible to mix one or more poorly soluble components with one component with a negative enthalpy of solution or one poorly soluble component with one or more components with a negative enthalpy of dissolution. Preferably, one poorly soluble component is co-lyophilized with one component with negative enthalpy of solution.

Suitable components with negative enthalpy of solution are all components which are no poorly soluble components and whose concentration in the composition is high enough so that they are present in an amount sufficient to be mixed with the poorly soluble component. Examples are inorganic salts like CaCl₂, MgSO₄ or CaO, bases like sodium hydroxide or potassium hydroxide as well as amino acids like proline. It has been found that proline is suitable for many applications, especially for preparing mixed particles with poorly soluble amino acids. If a shift of the pH might additionally positively influence the dissolution of a component, the bases sodium hydroxide or potassium hydroxide are preferably used. This is e.g. the case for improving the dissolution of tyrosine.

Preferably, the amount of components with negative enthalpy of solution are present in the mixed particles in an amount so that the amount of negative enthalpy of solution is at least double the amount of positive enthalpy of solution.

Preferably, the mixed particles only contain the one or more poorly soluble components whose solubility shall be improved and the one or more components with a negative enthalpy of solution who due to energy transfer support the dissolution of the poorly soluble components. The mixed particles preferably do not contain any or at least not more than 10% w/w of components which either do not have a negative enthalpy of solution or which are not poorly soluble, like sodium chloride or glucose. Though components like glucose or sodium chloride are easily soluble, their presence in the mixed particles would hinder the direct contact between the poorly soluble components and the components with a negative enthalpy of solution and thus the transfer of energy.

It is obvious that for improving the solubility of a dry mixture with a given composition the one or more components with a negative enthalpy of solution that are combined with the poorly soluble components to form the mixed particles are selected from components that are also present in the original composition of the dry mixture. Also the amount of such components is chosen such that it corresponds to the original amount present in the dry mixture. Otherwise the composition of the mixture would be changed. Consequently, the method of the present invention can only be used for dry mixtures comprising one or more components with a negative enthalpy of solution if it is aimed to improve the solubility without changing the overall composition of the dry mixture. Otherwise the composition of the dry mixture would have to be changed by adding one or more components with a negative enthalpy of solution that have not been present in the mixture before. Typically, for nutrient compositions and cell culture media this is not a critical issue as such dry mixtures comprise a sufficient number and amount of components with a negative enthalpy of solution.

After generating the mixed particles by e.g. dry compaction, wet granulation, lyophilisation or spray drying, the resulting mixed solid may then preferably be milled, e.g. in a ball mill, to generate particles of homogenous size. The resulting particles typically have a particle size below 200 μm. Preferred are particle sizes below 100 μm. Favourable particle sizes are between 15 μm and 100 μm.

The mixed particles can then be subjected to analytical methods to determine the concentration of the components in the particle. Examples of suitable methods are ICP-OES, UPLC (e.g. for amino acids), NMR, IC or LC-MS.

The final mixed particles can then be stored or used for the production of dry mixtures like cell culture media.

For the latter, a suitable amount of the mixed particles is mixed with the other components of the cell culture medium. It is also possible to generate two or more types of mixed particles and mix two or more types of mixed particles with the other components of the cell culture medium. The mixing of the components is known to a person skilled in the art of producing dry powdered cell culture media. Preferably, all components are thoroughly mixed so that all parts of the mixture have nearly the same composition. The higher the uniformity of the composition, the better the quality of the resulting medium with respect to homogenous cell growth.

Afterwards, the mixture is preferably milled.

The milling can be performed with any type of mill suitable for producing powdered cell culture media. Typical examples are ball mills, pin mills, fitz mills or jet mills. Preferred is a pin mill, a fitz mill or a jet mill, very preferred is a pin mill.

A person skilled in the art knows how to run such mills.

The present invention is further directed to a dry mixture, preferably a dry powder cell culture medium, comprising at least one mixed particle. Such medium is obtainable by the process according to the present invention.

Preferably, the dry mixture comprises 1 to 10 different mixed particles. Especially preferred are mixed particles comprising proline and one or more of valine, leucine, isoleucine and phenylalanine. Most preferred are particles only made of amino acids.

For use of the powdered dry mixtures a solvent, preferably water (most particularly distilled and/or deionized water or purified water or water for injection) or an aqueous buffer is added to the dry mixtures and the components are mixed until the dry mixture is totally dissolved in the solvent.

The solvent may also comprise saline, soluble acid or base ions providing a pH range of 1.0-14.0, stabilizers, surfactants, preservatives, and alcohols or other polar organic solvents.

In case of e.g. cell culture media, it is also possible to add further substances like buffer substances for adjustment of the pH, fetal calf serum, sugars etc., to the mixture of the cell culture medium and the solvent. The resulting liquid cell culture medium is then contacted with the cells to be grown or maintained.

The present invention is thus further directed to a process for culturing cells by

a) providing a cell culture medium comprising mixed particles, preferably mixed particles comprising proline and one or more of valine, leucine, isoleucine and phenylalanine.

b) mixing said cell culture medium with water or an aqueous buffer

c) mixing the cells to be cultured with the cell culture medium of step b) in a bioreactor

d) incubating the mixture of step c)

A bioreactor is any container, vessel, bag or tank in which cells can be cultured. Incubation is typically done under suitable conditions like suitable temperature, osmolality, aeration, agitation etc. A person skilled in the art is aware of suitable incubation conditions for supporting and/or maintaining the growth/culturing of cells.

By using the mixed particles for the production of dry mixtures the overall amount of the mixture components and thus the overall mixture composition remains the same as outlined in the recipe, but as the poorly soluble components are combined with the components with a negative enthalpy of solution, the overall solubility of the dry powder mixture is significantly higher compared to a dry powder mixture prepared without the use of mixed particles. By using the method of the invention, typically, the solubility of the poorly soluble component can be increased by at least 50%. Preferably, it is increased by at least 100%, which means that by using mixed particles at least the double amount of a poorly soluble component is soluble in the same liquid composition compared to the use of the poorly soluble substance as such.

The present invention is further illustrated by the following examples, however, without being restricted thereto.

The entire disclosure of all applications, patents, and publications cited above and below, as well as the corresponding European patent application EP 16181691.3 filed on Jul. 28, 2016, are hereby incorporated by reference.

### EXAMPLES

The following examples represent practical applications of the invention.

Composition of DMEM F12 cell culture medium (Sigma Aldrich article number D2906), dry powder, one-fold, composition in [g/L]:

Inorganic Salts

CaCl₂: 0.1166

CuSO₄.5H₂O 0.0000013

Fe(NO₃)₃.9H₂O 0.00005

FeSO₄.7H₂O 0.000417

MgCl₂.6H₂O 0.0612

MgSO₄ 0.04884

KCl 0.3118

NaCl 6.996

Na₂HPO₄ 0.07102

NaH₂PO₄ 0.0543

ZnSO₄.7H₂O 0.000432

Amino Acids

L-Alanine 0.00445

L-Arginine.HCl 0.1475

L-Asparagine.H₂O 0.0075

L-Aspartic Acid 0.00665

L-Cystine.2HCl 0.01756

L-Cysteine.HCl.H₂O 0.03129

L-Glutamic Acid 0.00735

L-Glutamine 0.365

Glycine 0.01875

L-Histidine.HCl.H₂O 0.03148

L-Isoleucine 0.05447

L-Leucine 0.05905

L-Lysine.HCl 0.09125

L-Methionine 0.01724

L-Phenylalanine 0.03548

L-Proline 0.01725

L-Serine 0.02625

L-Threonine 0.05345

L-Tryptophan 0.00902

L-Tyrosine.2Na.2H₂O 0.05579

L-Valine 0.05285

Vitamins

D-Biotin 0.0000035

Choline Chloride 0.00898

Folic Acid 0.00266

myo-Inositol 0.0126

Niacinamide 0.00202

D-Pantothenic Acid.½Ca 0.00224

Pyridoxal.HCl 0.002

Pyridoxine.HCl 0.000031

Riboflavin 0.000219

Thiamine.HCl 0.00217

Vitamin B12 0.00068

Other

D-Glucose 3.15

HEPES 3.5745

Hypoxanthine 0.0021

Linoleic Acid 0.000042

Putrescine.2HCl 0.000081

Pyruvic Acid.Na 0.055

DL-Thioctic Acid 0.000105

Thymidine 0.000365

ADD

NaHCO₃ 1.2

Material and Methods

Material

| | | | |
|---|---|---|---|
| | | Molecular | Cas. |
| Material | Manufacturer | weight [g/mol] | No. |

Amino Acids:

| | | | |
|---|---|---|---|
| L-leucine | Merck KGaA, | 131.17 | 61-90-5 |
| | Darmstadt | | |
| L-proline | Merck KGaA, | 115.13 | 147-85-3 |
| | Darmstadt | | |

Solutions:

| | | | |
|---|---|---|---|
| Destilled Water | Merck KGaA, | 18.02 | 7732- |
| (MilliQ) | Darmstadt | | 18-5 |

2. Methods

2.1.1 Spray Drying

2.1.1.1 BÜCHI Mini Spray DRYER B-290

To spray dry a solution of two substances the BÜCHI Mini Spray DRYER B-290 was used. In order to achieve a spray dried material, heated air is pumped with a pressure pump through a two-fluid nozzle cap (0.7 mm). The solvent (containing the product as solution or suspension) is additionally pumped into the nozzle and is sprayed in the drying chamber via an atomizer, which dispersed the solvent in small droplets due to the hot compressed air. Inside the drying chamber, the droplets vaporize through the preheated air. The remaining moistened air and the product powder leave the drying chamber through a cyclone, where the product settles down in a collection vessel. To insure a dry product the moist air and moist particles are evacuated by means of an exhaust fan, which filters the excess. A short drying time and low drying temperatures enables drying of heat sensitive products.¹⁷ To insure small, dry particles the outlet temperature needs to be optimized to its lowest possible value, which still assures a dry product and avoids an adhesion of particles and clump formation.¹⁸

Experimental Settings:
The Mini Spray DRYER was set on a Q-Flow of 40 m³/h.
An inlet temperature of 130° C. was set, which led to an outlet Temperature of 82° C.¹⁹
A peristaltic pump was adjusted to a pumping rate of 8 mL per min with constant aspiration of 100% of the solvent.
The nozzle cleaner was set to 5 times a min.

2.1.2 Co-lyophilisation—General Procedure

Variant 1:

The poorly soluble component is dissolved in ultrapure water (e.g. Milli-Q water) with stirring and if necessary at elevated temperature.

The component the negative enthalpy of solution is also dissolved in ultrapure water (e.g. Milli-Q water).

Both solutions are combined under stirring.

Variant 2:

The carrier component is dissolved in ultrapure water (e.g. Milli-Q water).

The poorly soluble component is added with stirring. The mixture is agitated and if necessary also heated until dissolution of the poorly soluble component.

In both variants, the concentration of the components, the dissolutions times and the temperatures need to be adjusted to the components to be dissolved. A person skilled in the art is able to do so.

For lyophilisation, the solution prepared according to variant 1 or 2 is poured in suitable containers (e.g. Lyoguards) and put in the freeze-dryer. Depending on the composition of the solution, it is cooled to about −45° C. within 4 to 12 hours. The condensator is cooled to −90° C. Then vacuum is applied (0.18 mbar) and the solution is dried at −45° C. for 6 hours. Then, during drying, the temperature is adjusted to about −30° C. for about 24 hours, to −20° C. for another 24 hours and finally to about 0° C. for another 12 hours. Final drying is performed under minimal vacuum (0.003 mbar) at a temperature of about 20° C. for 4 to 12 hours.

The time and the temperature gradient can be adjusted depending on the volume of solution to be dried.

2.2 Measurement of Dissolution Enthalpy

2.2.1 TAM IV Microcalorimeter by TA Instruments

The TAM IV Microcalorimeter was used to detect temperature changes of solutions with an accuracy of 0.001 K, which evolves from the dissolution enthalpy of the substances being tested.

Different formulations of amino acids are placed in an ampoule, surrounded by aqueous solvent. After the ampoule has been ruptured, the temperature change is analyzed.

Experimental Setup:
250 mg of each sample were filled into a 1 mL crushing ampoule at ambient conditions without any pre-treatment.
The ampoules were closed with a silicone stopper and wax and inserted into the stirrer of the solution calorimeter and immersed into 100 mL water.
The system was then equilibrated at 25° C. while stirring at 500 rpm before the ampoule was broken.
Calibrations with a heat pulse of 2.5 J at a power of 250 mW were performed before and after the break.
For data evaluation, the slope of the baseline before breaking the sample was set to zero by differentiation of the curve, shift to zero and re-integration.

2.3 Quantitative Analysis of Amino Acids

2.3.1 Ultra-Performance Liquid Chromatography (UPLC):

The UPLC was used as an analytical technique for the identification and quantification of amino acids in solutions. Amino acids were derivatized using a derivatization kit and then separated by liquid chromatography. The amino acids were identified according to their retention times compared to a standard (Zimmer A, Mueller R, Wehsling M, Schnellbaecher A, Hagen J Von. Improvement and simplification of fed-batch bioprocesses with a highly soluble phosphotyrosine sodium salt. J Biotechnol. 2014;186:110-118. doi:10.1016/j.jbiotec.2014.06.026.)

3.1 Spray Dried Multicomponent Amino Acids

The dissolution behavior of adjacent molecules and the hydrophobicity effects on the energies of solution are studied via calorimetry methods and are particularly important for studying thermodynamic properties of such systems.²⁰

For spray dried multicomponent amino acids the enthalpy of solution measurement have been carried out on a special ampule calorimeter equipped with an isothermal shell. In the course of the calorimetry measurement, the stability of the temperature-control system was maintained constant to within 10⁻⁴K. All experiments were performed three times and were reproducible within 5%.

FIGS. 1 and 2 show the disssolution of proline and leucine in form of the relative temperature. For the enthalpy of solution of L-proline a strong increase in the temperature is observed followed by a continous decrease (FIG. 1). The enthalpy of solution of L-leucine represents a steady decrease in the temperature (FIG. 2).

The enthalpy of solution of mixed L-proline and L-leucine powder in the ratio 2:1 reveals a strong increase in temperature followed by a steady decrease (FIG. 3). The temperature change of the same amino acids composition spray dried causes a small temperature decrease and than commutes along the base line (FIG. 4).

### SUMMARY OF RESULTS

1. Energy is released or absorbed by substances as they dissolve.

2. This energy, called dissolution enthalpy, can be transferred from an energy releasing substance to an energy absorbing one, for example from proline to leucine

3. The phenomenon can be enhanced by coupling the substances for example by spry drying.

4. For proline and leucine, a ratio of 2:1 was found to be most efficient for the energy transfer.

5. Particle size distribution of the spray dried material did not change. Therefore the effect of particle size can be excluded in the improvement of solubility.

4. Cell Culture Media Production

The above mixed particles are used for the preparation of a chemically defined cell culture media for Chinese hamster ovary cells.

By using the mixed particles the overall amount of components still remains the same as outlined in the recipe.

All ingredients of the medium including the mixed particles are mixed, and milled using a dosage snail and a pin mill. In the dosage snail the ingredients are treated with liquid nitrogen.

The milling is performed under the following conditions:

Temperature—mill: 10° C.

Oxygen level: below 10% absolute

Rpm—Mill: up to 2800 1/min

Blending time: 30 min

Rpm dosage snail: 40 1/min

The resulting powdered cell culture medium is suitable for the culture of CHO (Chinese Hamster Ovary) cells.

1. A method for improving the solubility of a dry mixture with a given composition by
a) identifying the one or more poorly soluble components in the said dry mixture
b) preparing mixed particles comprising at least one poorly soluble component and one component with a negative enthalpy of solution
c) preparing the said dry mixture with improved solubility by mixing the one or more mixed particles generated in step b) with the other components of dry mixture

2. Method according to claim 1, characterized in that the one or more poorly soluble components are compounds with a solubility of less than 20 g/l in an aqueous solution comprising more than 100 g/l of dissolved components at 25° C.

3. Method according to claim 1, characterized in that the one or more components with a negative enthalpy of solution are selected from the following group:
proline
Calcium Chloride, anhydrous
Magnesium Sulfate, anhydrous
Calcium Oxide, anhydrous
Sodium Hydroxide
Potassium Hydroxide or mixtures thereof.

4. Method according to claim 1, characterized in that the component with a negative dissolution enthalpy is proline.

5. Method according to claim 1, characterized in that the one or more poorly soluble components are selected from the group of leucine, isoleucine, valine and phenylalanine.

6. Method according to claim 1, characterized in that in step b) the preparation of the mixed particles is performed by spray drying, lyophilisation or wet granulation.

7. Method according to claim 1, characterized in that in step b) the preparation of the mixed particles is performed by spray drying.

8. Method according to claim 1, characterized in that the dry mixture is a dry powder cell culture medium.

9. Method according to claim 1, characterized in that the composition of the mixed particles is such that the amount of the negative enthalpy of solution is double or more than the amount of the positive enthalpy of solution of the poorly soluble component.

10. Method according to claim 1, characterized in that the mixed particles prepared in step b) comprise proline in combination with one or more of the components selected from leucine, isoleucine, valine and phenylalanine.

11. Method according to claim 1, characterized in that 90% or more (w/w) of the mixed particles consist of the one or more poorly soluble components and the one or more components with a negative enthalpy of solution.

12. A dry mixture comprising one or more mixed particles containing at least one component which is poorly soluble in water and at least one component with a negative dissolution enthalpy.

13. Dry mixture according to claim 12, characterized in that the dry mixture is a dry powder cell culture medium.

14. Dry mixture according to claim 12, characterized in that the dry mixture comprises mixed particles comprising proline in combination with one or more of the components selected from leucine, isoleucine, valine and phenylalanine.

15. Dry mixture according to claim 12, characterized in that 90% or more (w/w) of the mixed particles consist of the one or more poorly soluble components and the one or more components with a negative enthalpy of solution.
